(19) United States
(12) Patent Application Publication
(10) Pub. No.: US 2006/0134042 A1
(43) Pub. Date: Jun. 22, 2006

(54) Hair shaping composition comprising at least one tetramethylguanidine

(76) Inventors: Gerard Malle, Villiers S/Morin (FR); Damien Lavergne, Paris (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/989,621

(22) Filed: Nov. 17, 2004

Related U.S. Application Data
(60) Provisional application No. 60/562,294, filed on Apr. 15, 2004.

(30) Foreign Application Priority Data
Nov. 18, 2003 (FR) 0350853

(51) Int. Cl. A61K 8/43; A61K 8/19

(52) U.S. Cl. 424/70.2

(57) ABSTRACT

The present disclosure relates to a ready-to-use composition for permanently shaping keratin fibers comprising, as permanent shaping agent, at least one tetramethylguanidine, a kit comprising compartments comprising the contents of the ready-to-use composition, and also to a process for using this composition.

This application claims benefit of U.S. Provisional Application No. 60/562,294, filed Apr. 15, 2004.

The present disclosure relates to a ready-to-use cosmetic composition for permanently shaping keratin fibers, comprising, as a permanent shaping agent, tetramethylguanidine. The present disclosure is also directed to a kit comprising compartments to be placed in contact to form the ready-to-use composition, and also to a process using the composition.

As used herein, the expression “tetramethylguanidine” includes 1,1,3,3,-tetramethylguanidine (Registry Number [80-70-6] and the organic or mineral salts thereof.

Also as used herein, the term “keratin fibers” means fibers of human or animal origin such as head hair, other body hairs, the eyelashes, wool, angora, cashmere or fur. Although the present disclosure is not limited to particular keratin fibers, reference will nevertheless be made more frequently to head hair.

Further as disclosed herein, the term “permanent shaping” includes the curling, permanent waving, and/or setting of Caucasian, Asiatic or North African hair.

As used herein, the expression “between x % and y %” means ranging from x to y %, the limits x and y being included.

In general, two techniques are used to permanently reshape the hair. They are based on breaking the disulfide bonds present in keratin (cystine):

The first technique comprises, in a first step, in opening the disulfide bonds by means of a composition comprising a reducing agent, and then, after having, for example, rinsed the hair, in reconstituting, in a second step, the disulfide bonds by applying to the hair, which has been placed under tension beforehand with curlers or the like or shaped or smoothed out by other means, an oxidizing composition also known as a fixing composition, so as to give the head of hair the desired shape. This technique can make it possible either to permanently shape the hair, or to relax it, decurl it or straighten it out.

The second technique comprises performing a lanthionization operation, using a composition comprising a base belonging to the hydroxide family. This leads to replacement of the disulfide bonds (—CH₂—S—S—CH₂—) with lanthionine bonds (—CH₂—S—CH₂—). This lanthionization operation can involve two consecutive chemical reactions:

The first reaction comprises a beta-elimination on the cystine brought about by a hydroxide ion, leading to the breaking of the bond and the formation of dehydroalanine.

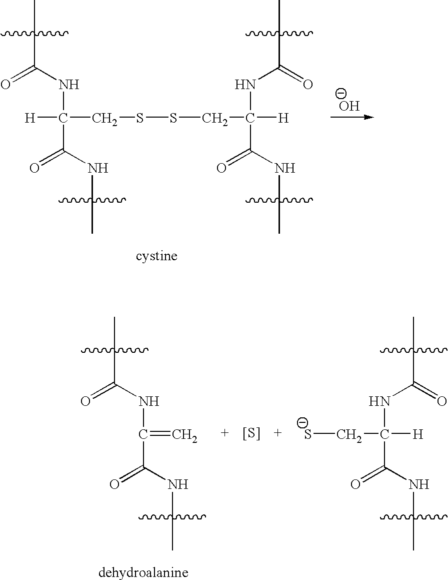

The second reaction is a reaction of the dehydroalanine with a thiol group. For example, the double bond of the dehydroalanine formed can be a reactive double bond. It can react with the thiol group of the cysteine residue that has been released to form a new bond, referred to as a lanthionine bridge or bond or residue.

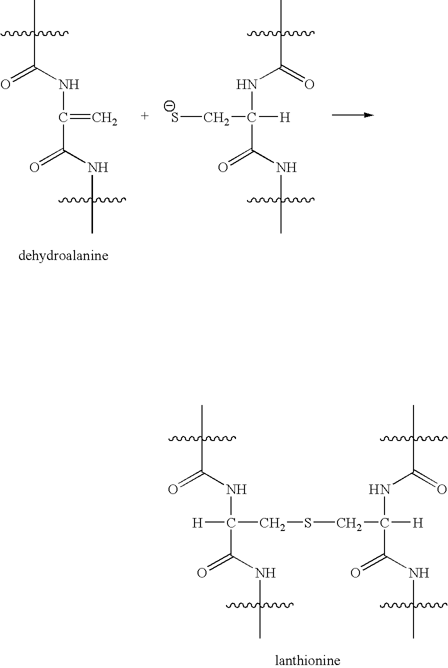

Relative to the first technique using a reducing agent, this lanthionization technique does not require a fixing step, since the formation of the lanthionine bridges is irreversible. It can thus take place in a single step and can make it possible either to make the hair wavy or to relax it, decurl it or straighten it out. However, it is mainly used to relax naturally frizzy or curly hair.

For the first technique, the reducing compositions that can generally be used for the first step of a permanent-waving or relaxing operation comprise thiols, sulfites or bisulfites as reducing agent. These agents can be used in an essentially aqueous medium at concentrations ranging from 0.5 to 1M to obtain good opening of the disulfide bonds. Among the thiols, those commonly used are thioglycolic acid, cysteamine, glyceryl monothioglycolate, thiolactic acid and cysteine. Thioglycolic acid, for example, can be efficient at reducing the disulfide bonds of keratin at alkaline pH, for instance, in the form of ammonium thioglycolate, and is the product most commonly used in permanent-waving (hair waving). However, it has been found that thioglycolic acid should be used in a sufficiently basic medium (for example, at a pH ranging from 8.5 to 9.5) if it is desired to obtain curling of sufficient intensity. Besides the drawback of releasing an unpleasant odor, which can require the use of more or less efficient fragrances to mask the odors, the use of a thiol at alkaline pH can also lead to degradation of the fiber as well as impairment of artificial colorations.

Sulfites or bisulfites are mainly used for relaxing. They can have drawbacks similar to thiols, with lower efficacy. Thiols and sulfites (or bisulfites) can also have the drawback of having poor stability in aqueous solution.

In general, the durability of the reshaping effects obtained with thiols and/or sulfites by reduction of disulfides followed by fixing is considered to be inferior to that which may be obtained via the lanthionization technique.

For the second technique, the compositions generally used to perform the lanthionization comprise at least one hydroxide base, such as sodium hydroxide, guanidinium hydroxide and lithium hydroxide. The lanthionization active agents, which make it possible to open the disulfide bonds via a beta-elimination mechanism, can generally be used in water-oil emulsions at concentrations ranging from 0.4M to 0.6M, by leaving them to act generally for a period of time ranging from 10 to 15 minutes at room temperature. Sodium hydroxide is the agent most commonly used. Guanidinium hydroxide is now the preferred compound for many compositions. These two hydroxides, i.e., sodium hydroxide and guanidinium hydroxide, are the two main agents used for relaxing and/or decurling naturally frizzy hair. They can have several advantages over ammonium thioglycolate and/or sulfites, for example, an absence of unpleasant odor, the fact that only one implementation step is required (shorter treatment time), and can have a greater durability and efficacy of the reshaping of the hair.

However, these hydroxides can have the major drawback of being caustic. This causticity can affect the scalp by causing irritation that on occasion is severe. This may be partially overcome by the prior application to the scalp of a greasy protective cream often referred to as “base” or “base cream,” the word “base,” as used in this context, not having the meaning of a basic agent in the chemical sense. When the protective cream is combined with the hydroxide in a single composition, this is generally referred to as a “no-base” composition, as opposed to the above name. This “no-base” technology is more frequently used, in part because it is more convenient.

The causticity of the hydroxides can also affect the state of the hair by possibly giving it a coarse feel and/or making it much more fragile, this fragility possibly going as far as fraying, breaking or even dissolution of the hair if the treatment is prolonged. In certain cases, hydroxides can also cause decoloration of the natural color of the hair.

Formulations comprising sodium hydroxide are generally referred to as “lye relaxers” and those not comprising it are known as “no-lye relaxers”.

The main “no-lye” relaxing formulations use guanidinium hydroxide. Since guanidinium hydroxide can be unstable, it is generated extemporaneously by mixing guanidine carbonate and a source of very sparingly soluble hydroxide such as calcium hydroxide. The reaction between these two compounds leads to the formation of guanidinium hydroxide and calcium carbonate, which precipitates in the composition. The presence of this precipitate may make the final rinsing of the hair much more difficult and can leave on the hair and scalp mineral particles that give it a coarse feel and an unaesthetic appearance resembling dandruff. The recent success of guanidinium hydroxide (“no-lye”) over sodium hydroxide (“lye”) appears to arise from better relaxing efficacy and better skin tolerance. However, the technologies using bases of the hydroxide family remain very aggressive for the hair and the scalp and may require very strict control of the application time to avoid excessive irritation and impairment of the hair that may go as far as breaking. The aggressiveness associated with the causticity of hydroxides is one reason these compositions for the lanthionization of the hair are not currently used for permanent-waving (hair waving), but instead generally reserved for relaxing (hair straightening or hair relaxing).

Furthermore, hydroxides are known to be good agents for hydrolyzing amide functional groups (compare, for example, March's Advanced Organic Chemistry, 5th Edition, Wiley Interscience, New York, “Hydrolysis of Amides” pages 474 et seq), which thus lead to breaking of the peptide bonds by direct nucleophilic attack. It is thus possible that the impairments observed in the case of the hair and keratin materials in the broad sense are largely due to partial hydrolysis of the amide bonds of keratin.

There is thus a real need, in the art of hair shaping, for compositions that are less aggressive to the hair and the skin.

Various studies have been performed with a view to simultaneously overcoming at least one of the drawbacks of using reducing agents (first technique) and/or of using hydroxides (second technique).

Accordingly, to replace thioglycolic acid, many reducing agents have been proposed, but thioglycolic acid in its ammonium thioglycolate form remains both the reference compound and the compound most widely used in cosmetic formulations, for permanently shaping the hair.

However, the use of reducing agents can lead to an insufficient durability for permanent shaping, which may make it necessary to frequently repeat the treatment and may lead inevitably to gradual degradation in the course of repeated applications. Moreover, the causticity of the hydroxides restricts their use solely to the relaxing field. Thus, what is needed is, as active ingredient for permanent shaping of the hair, a base which would be both effective and less aggressive than sodium hydroxide or hydroxides in the broad sense.

After extensive studies, it has now been discovered, entirely surprisingly and unexpectedly, that the first step of the lanthionization process can be performed with tetramethylguanidine. Excellent results in terms of intensity of permanent shaping, and/or in terms of cosmetic and mechanical qualities of the hair may thus be obtained, with a durability greater than that which can be obtained with the reference product for permanent shaping, i.e., ammonium thioglycolate.

Accordingly, one aspect of the present disclosure is a cosmetic composition for permanently shaping keratin fibers comprising, in a cosmetically acceptable medium, at least one tetramethylguanidine, the cosmetically acceptable medium and the concentration of the at least one tetramethylguanidine being chosen such that the at least one tetramethylguanidine is reactive with the cystines of the keratin fibers, via a beta-elimination reaction, to produce dehydroalanine and lead to the formation of lanthionine, to obtain keratin fibers having curls with a diameter ranging from 0.2 cm to 3 cm, in a permanent shaping time of less than 60 minutes.

For example, the permanent shaping time can be less than 40 minutes, such as less than 30 minutes.

When the compositions according to the present disclosure are intended for a keratin fiber permanent shaping process, the tetramethylguanidine can be present, for example, in a molar concentration ranging from 0.01 M to 1.5M, which corresponds to an amount ranging from 0.115% to 17.25% by weight, relative to the total weight of the composition, and for instance in a concentration ranging from 0.05M to 1M, which corresponds to an amount ranging from 0.575% to 11.5% by weight, relative to the total weight of the composition.

The pH of the compositions according to the present disclosure can range, for example, from 9.6 to 14, such as from 11 to 13.

In one embodiment of the present disclosure, for example, the at least one tetramethylguanidine is the only permanent shaping active agent.

The compositions according to the present disclosure may also comprise at least one known reducing agent, for instance thioglycolic acid, thiolactic acid and ester and amide derivatives thereof, for instance glyceryl monothioglycolate, cysteamine and its C1-C4 acyl derivatives such as N-acetylcysteamine or N-propionylcysteamine, cysteine, N-acetylcysteine, thiomalic acid, pantethine, 2,3-dimercaptosuccinic acid, sulfites and/or bisulfites of an alkali metal or alkaline-earth metal, the N-(mercaptoalkyl)-ω-hydroxyalkylamides described in European Patent Application No. EP-A-354 835, the N-mono- or N,N-dialkylmercapto-4-butyramides described in European Patent Application No. EP-A-368 763, the aminomercaptoalkylamides described in European Patent Application No. EP-A-432 000, the N-(mercaptoalkyl)succinamic acid and N-(mercaptoalkyl)succinimide derivatives described in European Patent Application No. EP-A-465 342, the alkylamino mercaptoalkylamides described in European Patent Application No. EP-A-514 282, the azeotropic mixture of 2-hydroxypropyl thioglycolate and of (2-hydroxy-1-methyl)ethyl thioglycolate described in French Patent Application No. FR-A-2,679,448, the mercaptoalkylaminoamides described in French Patent Application No. FR-A-2,692,481, the N-mercaptoalkylalkanediamides described in European Patent Application No. EP-A-653 202 and the formamidinesulfinic acid derivatives described in PCT Application No. PCT/US 01/43124.

When the compositions as disclosed herein comprise at least one reducing agent, this agent is can be present in an amount, for example, of less than or equal to 20% by weight, for instance ranging from 0.1% to 10% by weight, relative to the total weight of the composition.

The compositions according to the present disclosure may also comprise at least one hydroxide chosen, for example, from alkali metals, alkaline-earth metals, transition metals, and organic hydroxides such as sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide and quaternary ammonium hydroxides.

When the compositions as disclosed herein comprise at least one hydroxide it can be present, for example, in an amount ranging from 0.01% to 3.5% by weight, for instance, ranging from 0.05% to 1.5% by weight, relative to the total weight of the composition.

In one embodiment of the compositions according to the present disclosure, for example, these compositions comprise 0% of hydroxide bases, including those chosen from alkali metals, alkaline-earth metals, transition metals, and organic hydroxides.

The compositions as disclosed herein may also comprise at least one surfactant chosen from those of nonionic, anionic, cationic and amphoteric type, such as alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters and other nonionic surfactants of the hydroxypropyl ether type.

When the compositions comprise at least one surfactant, it can be present in an amount less than or equal to 30% by weight, for instance ranging from 0.5% to 10% by weight, relative to the total weight of the composition.

In order to improve the cosmetic properties of the hair and/or to attenuate or avoid its degradation, the composition may also comprise at least one treating agent chosen from those of cationic, anionic, nonionic and amphoteric nature.

Among the treating agents that may be used as disclosed herein, non-limiting mention may be made, for example, of those described in French Patent Nos. 2,598,613 and 2,470,596. It is also possible to use as treating agents volatile and non-volatile, linear and cyclic silicones and mixtures thereof, polydimethylsiloxanes, quaternized polyorganosiloxanes such as those described in French Patent Application No. 2,535,730, polyorganosiloxanes comprising aminoalkyl groups modified with alkoxycarbonylalkyl groups, such as those described in U.S. Pat. No. 4,749,732, polyorganosiloxanes such as the polyoxyalkyl polydimethylsiloxane copolymer of the dimethicone copolyol type, a polydimethylsiloxane comprising stearoxy end groups (stearoxy dimethicone), a dialkylammonium acetate polydimethylsiloxane or a polydimethylsiloxane polyalkylbetaine copolymer described in British Patent No. 2,197,352, polysiloxanes organomodified with mercapto or mercaptoalkyl groups, such as those described in French Patent No. FR1,530,369 and in European Patent Application No. EP 295 780, and also silanes such as stearoxytrimethylsilane.

The compositions according to the present disclosure may also comprise at least one other treating ingredient such as cationic polymers, for instance those used in the compositions of French Patent Nos. 2,472,382 and 2,495,931, or cationic polymers of the ionene type, such as those used in the compositions of Luxembourg Patent No. 83703, basic amino acids (such as lysine or arginine) or acidic amino acids (such as glutamic acid or aspartic acid), peptides and derivatives thereof, protein hydrolyzates, waxes, swelling agents and penetrating agents or agents for reinforcing the efficacy of the reducing agent, such as the SiO₂/PDMS (polydimethylsiloxane) mixture, dimethylisosorbitol, urea and its derivatives, pyrrolidone, N-alkylpyrrolidones, thiamorpholinone, alkylene glycol or dialkylene glycol alkyl ethers, for instance propylene glycol monomethyl ether, dipropylene glycol monomethyl ether, ethylene glycol monoethyl ether and diethylene glycol monoethyl ether, C3-C6 alcanediols such as, for example, 1,2-propanediol, 1,3-propanediol and 1,2-butanediol, glycerol, 2-imidazolidinone, and also other compounds such as fatty alcohols, lanolin derivatives, active ingredients such as pantothenic acid, agents for preventing hair loss, antidandruff agents, thickeners, suspending agents, sequestering or complexing agents, opacifiers, sunscreens, fragrances and preserving agents.

The basic composition according to the present disclosure can be essentially in aqueous form, for example in the form of a lotion, thickened or otherwise, of a cream or of a gel.

The basic composition as disclosed herein may also comprise at least one solvent such as, for example, ethanol, propanol, isopropanol, and butanol, or alternatively glycerol, in an amount of less than or equal to 20% by weight, relative to the total weight of the composition.

The cosmetically acceptable medium of the basic compositions as disclosed herein can be, for example, water or a hydroalcoholic solution of water and at least one C2-C8 alcohol, such as ethanol or isopropanol.

The compositions according to the present disclosure may also be in the form of a thickened cream so as to hold the hair in the final desired shape. These creams are made in the form of “heavy” emulsions, for example based on glyceryl stearate, glycol stearate, self-emulsifying waxes or fatty alcohols.

Liquids or gels comprising thickeners, such as carboxyvinyl polymers or copolymers that “stick” the hairs together and hold them in the desired shape during the leave-in time, may also be used.

The compositions according to the present disclosure may also comprise at least one adjuvant chosen from silicones in soluble, dispersed and microdispersed form; nonionic, anionic, cationic and amphoteric surfactants; ceramides, glycoceramides and pseudoceramides; vitamins and provitamins including panthenol; plant, animal, mineral and synthetic oils; waxes other than ceramides, glycoceramides and pseudoceramides; water-soluble and liposoluble, silicone-based and non-silicone-based sunscreens; nacreous agents and opacifiers; sequestering agents; plasticizers; solubilizers; acidifying agents; mineral and organic thickeners; antioxidants; hydroxy acids; penetrating agents; fragrances and preserving agents.

The present disclosure also relates to a kit comprising at least two compartments, wherein at least one of the compartments comprises a composition for permanently shaping keratin fibers comprising, in a cosmetically acceptable medium, at least one tetramethylguanidine, wherein the at least one tetramethylguanidine is reactive with the cystines of keratin fibers, via a beta-elimination reaction to produce dehydroalanine and lead to the formation of lanthionine, to obtain keratin fibers having curls with a diameter ranging from 0.2 cm to 3 cm, in a permanent reshaping time of less than 60 minutes.

In one embodiment of the present disclosure, the kit a also comprises at least one additional composition for caring for, conditioning, making up, removing makeup from, protecting, cleansing and/or washing keratin fibers.

The compositions of the kits according to the present disclosure are packaged in separate compartments, containers or devices, optionally accompanied by suitable, identical or different application means, such as fine brushes, coarse brushes or sponges.

Another aspect of the present disclosure concerns a process for permanently shaping keratin fibers using a cosmetic composition comprising, in a cosmetically acceptable medium, at least one tetramethylguanidine, wherein the cosmetically acceptable medium and the concentration of the at least one tetramethylguanidine are chosen such that the at least one tetramethylguanidine reacts with cystines of the keratin fibers, via a beta-elimination reaction to produce dehydroalanine and lead to the formation of lanthionine, to obtain keratin fibers having curls with a diameter ranging from 0.2 cm to 3 cm in a permanent shaping time of less than 60 minutes.

For example, in the process as disclosed herein, the permanent shaping time can be less than 40 minutes, such as less than 30 minutes.

In the permanent shaping process according to the present disclosure, the composition can be applied, for example, to dry keratin fibers, such as hair, or keratin fibers that have been made wet beforehand, previously put in rollers or curlers with a diameter ranging from 2 mm to 30 mm, it being possible for the composition to be applied gradually while putting the keratin fibers (e.g., hair) in curlers; the composition is then left to act for a period of time ranging from 5 to 60 minutes, for example 5 to 40 minutes. After removing the rollers, the keratin fibers (e.g., hair) are rinsed thoroughly.

According to the present disclosure, after applying the composition as disclosed herein, the keratin fibers, such as a head of hair, may be, for example, subjected to a heat treatment by heating to a temperature ranging from 30° C. to 60° C. In practice, this operation may be performed using a hairstyling hood, a hair dryer, an infrared ray dispenser and/or other standard heating devices.

It is possible to use, as a method of both heating and shaping the keratin fibers, a hot iron at a temperature ranging from 60° C. to 220° C., such as ranging from 120° C. to 200° C.

Yet another aspect of the present disclosure concerns the use of tetramethylguanidine as an active agent for permanently shaping keratin fibers.

The present disclosure also relates to an active agent for permanently shaping keratin fibers, by means of a beta-elimination reaction to produce dehydroalanine and lead to the formation of lanthionine, comprising at least one tetramethylguanidine.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements.

The following examples are intended to illustrate the invention in a non-limiting manner. In the examples, unless otherwise indicated, the percentages and parts are expressed on a weight basis.

### EXAMPLE 1

A simplified permanent waving composition was prepared, comprising tetramethylguanidine, at a concentration of 0.8M in water, as the permanent waving active agent. The pH of the composition was 13.2. This composition was applied to Caucasian hair which had been put in curlers beforehand, for 15 minutes at a temperature of 30° C. The hair was rinsed and dried. The hair had beautiful curls and felt soft.

### EXAMPLE 2

A simplified permanent waving composition was prepared, comprising tetramethylguanidine, at a concentration of 0.5M in water, as the permanent waving active agent. The pH of the composition was 13.1. This composition was applied to Caucasian hair which had been put in curlers beforehand, for 30 minutes at a temperature of 30° C. The hair was rinsed and dried. The hair had beautiful curls and felt soft.

What is claimed is:

1. A composition for permanently shaping keratin fibers comprising, in a cosmetically acceptable medium, at least one tetramethylguanidine, wherein the cosmetically acceptable medium and the concentration of the at least one tetramethylguanidine are chosen such that the at least one tetramethylguanidine is reactive with the cystines of the keratin fibers, via a beta-elimination reaction, to produce dehydroalanine and lead to the formation of lanthionine, to obtain keratin fibers having curls with a diameter ranging from 0.2 cm to 3 cm, in a permanent reshaping time of less than 60 minutes.

2. The composition according to claim 1, wherein the permanent shaping time is less than 40 minutes.

3. The composition according to claim 2, wherein the permanent shaping time is less than 30 minutes.

4. The composition according to claim 1, wherein the at least one tetramethylguanidine is present in an amount ranging from 0.01M to 1.5M.

5. The composition according to claim 4, wherein the at least one tetramethylguanidine is present in an amount ranging from 0.05M to 1M.

6. The composition according to claim 1, wherein the pH ranges from 9.6 to 14.

7. The composition according to claim 6, wherein the pH ranges from 11 to 13.

8. The composition according to claim 1, wherein the composition does not comprise a base belonging to the hydroxide family

9. The composition according to claim 1, further comprising at least one adjuvant chosen from silicones in soluble, dispersed and microdispersed form; nonionic, anionic, cationic and amphoteric surfactants; ceramides, glycoceramides and pseudoceramides; vitamins and provitamins; plant, animal, mineral and synthetic oils; waxes other than ceramides, glycoceramides and pseudoceramides; water-soluble and liposoluble, silicone-based and non-silicone-based sunscreens; nacreous agents and opacifiers; sequestering agents; plasticizers; solubilizers; acidifying agents; mineral and organic thickeners; antioxidants; hydroxy acids; penetrating agents; fragrances and preserving agents.

10. A kit for permanently shaping keratin fibers comprising at least two compartments, wherein at least one of the compartments comprises a composition comprising, in a cosmetically acceptable medium, at least one tetramethylguanidine, which is reactive with the cystines of keratin fibers, via a beta-elimination reaction to produce dehydroalanine and lead to the formation of lanthionine, to obtain keratin fibers having curls with a diameter ranging from 0.2 cm to 3 cm, in a permanent reshaping time of less than 60 minutes.

11. The kit according to claim 10, further comprising, in at least one second compartment, at least one additional composition for caring for, conditioning, making up, removing makeup from, protecting, cleansing and/or washing keratin fibers.

12. A process for permanently shaping keratin fibers comprising
applying to the fibers a composition comprising, in a cosmetically acceptable medium, at least one tetramethylguanidine, wherein the cosmetically acceptable medium and the concentration of the at least one tetramethylguanidine are chosen such that the at least one tetramethylguanidine is reactive with the cystines of the keratin fibers, via a beta-elimination reaction to produce dehydroalanine and lead to the formation of lanthionine, to obtain keratin fibers having curls with a diameter ranging from 0.2 cm to 3 cm, in a permanent shaping time of less than 60 minutes.

13. The process for permanently shaping keratin fibers according to claim 12, further comprising, after applying the composition to the fibers, heating the fibers to a temperature ranging from 30° C. to 60° C.

14. The process for permanently shaping keratin fibers according to claim 12, comprising heating the keratin fibers with a hot iron at a temperature ranging from 60° C. to 220° C.

15. The process for permanently shaping keratin fibers according to claim 14, wherein the hot iron is at a temperature ranging from 120° C. to 200° C.

16. The process for permanently shaping keratin fibers according to claim 12, wherein the permanent shaping time is less than 40 minutes.

17. The process for permanently shaping keratin fibers according to claim 16, wherein the permanent shaping time is less than 30 minutes.

18. An active agent for permanently shaping keratin fibers comprising at least one tetramethylguanidine reactive with the cystines of keratin fibers via a beta-elimination reaction producing dehydroalanine and leading to the formation of lanthionine.
